(19) United States
(12) Patent Application Publication
(10) Pub. No.: US 2012/0184823 A1
(43) Pub. Date: Jul. 19, 2012

(54) Integrated health and entertainment management system for smart handheld device

(75) Inventor: Chung-Chieh Chen, Taichung City (TW)

(73) Assignee: Cycling & Health Tech Industry R & D Center

(21) Appl. No.: 12/930,750

(22) Filed: Jan. 14, 2011

(51) Int. Cl. A61B 5/00; A63B 71/00

(52) U.S. Cl. 600/301; 482/9

(57) ABSTRACT

An integrated health and entertainment management system for a smart handheld device contains a game controlling unit, an external operating signal receiving module, a data analyzing module, a feedback signal generating module, an external apparatus controlling unit; and a controlling signal transmitting module. The system interfaces the smart handheld device with an external exercise apparatus so as to allow a user who uses the smart handheld device when operating the exercise apparatus to perform personal health and entertainment management.

### BACKGROUND OF THE INVENTION

1. Technical Field

The present invention relates to an interactive tool in the form of a program for smart handheld devices, and more particularly, to an integrated health and entertainment management system to be installed in a smart handheld device for interfacing the smart handheld device with an external exercise apparatus so as to allow a user who uses the smart handheld device when operating the exercise apparatus to perform personal health and entertainment management.

2. Description of Related Art

With the gradual improvement of living quality, people nowadays enjoy the pleasure of having a big meal rich in high protein or calories or eating tons of over-seasoned food and usually lack of sufficient exercise that helps to keep physical balance, thus bringing a risk to the health. Even knowing that well-balanced dietary habit and steady moderate exercise are important to the maintenance of a vigorous body, people tend to indulge in relying on convenient electric transportation devices, such as elevators, cars, metro or subway, instead of walking on feet. However, thanks to the health awareness and amusement need among the public that gain more and more significance, a trend tracing back to exercise need of human beings drives a development of exercise equipments in combination with amusement. Therefore, more and more exercise equipments have been invented and introduced to meet the exercise and amusement needs.

Conventional exercise apparatuses have preset operating modes with some certain settings based on a few programmed exercise patterns, thus substantively limiting the apparatuses from meeting various individual needs. However, as described in a prior art, Taiwan Patent No. 220388 titled “Multi-Media Exercise Apparatus” has successfully overcome the dull and plain operating mode in conventional exercise apparatuses and thereby improved users' exercise experience. Said prior art incorporates a multi-media device in an existing exercise apparatus and encourages users to exercise their bodies along with interactive games, thus achieving better exercise efficiency. By virtue of the combination of the exercise apparatuses and the multi-media device, the prior art realizes fitness, exercise and entertainment effects, and thus enhancing the user's interest and incentive to use such apparatuses continually. In addition to the multi-media device, the prior art further comprises a communication device for updating the multi-media device with additional virtual reality programs available over the Internet, so as to increase entertaining effect and reduce dullness brought by repeating limited game modes.

Despite of foregoing advantages, the disadvantages existing in the prior arts are the limitation to the mobility and connectability in said apparatus. That is, the multi-media function is limited to support only one said apparatus. Besides, there are still many technical issues to be solved with regard to change in users' individual settings.

On the other hand, the introduction of smart handheld devices has opened a new leaf of the communication technology. The smart handheld devices supporting detection, storage and communication functions include, but are not limited to PDA mobile phones, I-PAD, I-Phone and other portable electronic communication equipments supplied with sensing devices such as G-SENSORs, gyroscopes, digital compasses and the like. Further strength of the smart handheld devices is the capacity of supporting third-party software, thus allowing the smart handheld devices to provide almost infinite functions. In brief, the smart handheld devices can be a portable mobile phone equipped with GPRS, Bluetooth, infrared transmission and WIFI wireless transmission, capable of installing or uninstalling application software and compatible with various local hardware equipments.

Therefore, the application of said encouraging and interactive game interface in the smart handheld devices in combination of health management is believed to be innovative and revolutionary to the way modern people keep fitness.

### SUMMARY OF THE INVENTION

In the light of above described herein, the present invention provides an integrated health and entertainment management system to be built in a smart handheld device so as to enhance exercise efficiency and provide an interactive, entertaining exercise manner by combining the smart handheld device that is portable, equipped with sensing function and excellent in communication, capable of connecting with various external exercise apparatuses, and encouraging the users to take exercise continually and thus facilitating physical health and disease prevention.

For fulfilling the above goals, by referring to the drawings, the present invention involves an integrated health and entertainment management system to be built in a smart handheld device. The integrated health and entertainment management system comprises a game controlling unit having a game interface module for providing an interface in a display unit of the smart handheld device; an external operating signal receiving module for receiving an operating signal from an external exercise apparatus, the operating signal being a resultant of real-time operation made by a user toward the external exercise apparatus; a data analyzing module that compares the operating signal with a default, then accordingly generates a comparison result, and transforms the comparison result into a real-time dynamic image of a target in said interface; a feedback signal generating module producing a feedback signal subject to the comparison result; and an external apparatus controlling unit including a processor module generating a controlling signal subject to a predetermined training program and adjusting the controlling signal according to the feedback signal to change the predetermined training program; and a controlling signal transmitting module sending out the controlling signal that drives the external exercise apparatus to execute the preset training program.

The implementation of the present invention can achieve at least following objectives.

The primary objective of the present invention is to provide the present invention that involves the integrated health and entertainment management system of the smart handheld device that is portable, equipped with sensing function and excellent in communication and capable of connecting with various external exercise apparatuses, so as to transform the user's operation toward the external exercise apparatus into the corresponding motion of a target in the interface and to encourage the user to take exercise continually through interactive audio and video image or sound effects and thus facilitating physical health and disease prevention.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of an illustrative embodiment when read in conjunction with the accompanying drawings, wherein:

FIG. 1 is a structural diagram of one embodiment of an integrated health and entertainment management system built in a smart handheld device according to the present invention;

FIG. 2 is a structural diagram of another embodiment of the integrated health and entertainment management system built in the smart handheld device according to the present invention;

FIG. 3 is a structural diagram of another embodiment of the integrated health and entertainment management system built in the smart handheld device according to the present invention;

FIG. 4 is a diagram of the integrated health and entertainment management system built in the smart handheld device according to the present invention, and it shows signal correlation between said system and external apparatuses or devices;

FIG. 5 is a diagram of one embodiment of the integrated health and entertainment management system built in the smart handheld device according to the present invention, and it shows the connection between said device and applicable external apparatuses; and

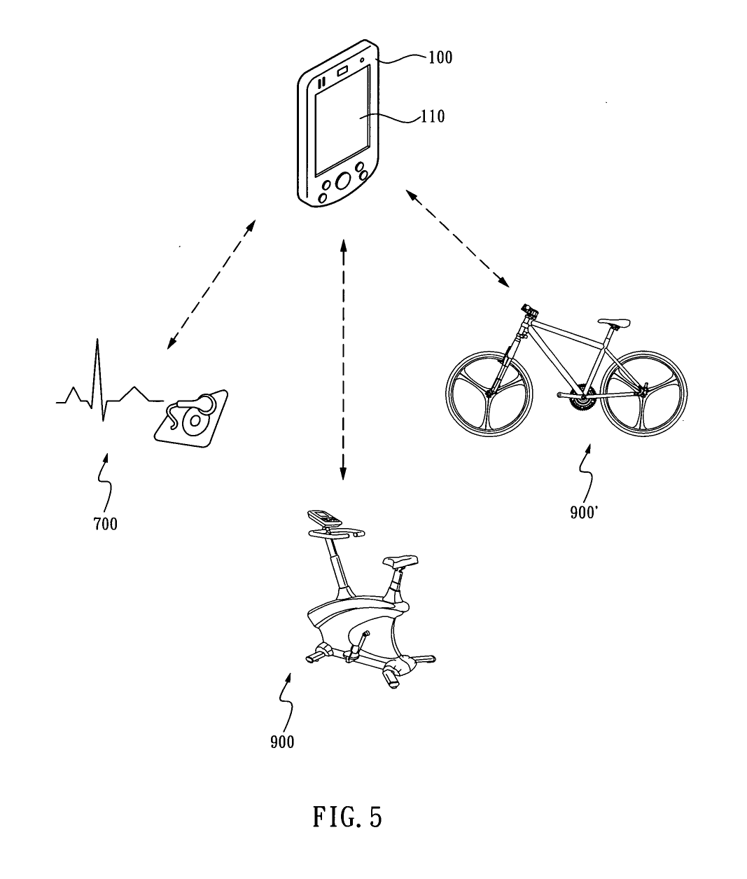

FIG. 6 is a diagram of the other embodiment of the integrated health and entertainment management system built in the smart handheld device according to the present invention, and it shows the connection between said device and applicable external apparatuses.

### DETAILED DESCRIPTION OF THE PREFERRED EMBODIMENT

For the convenience of explanation of the concept of subject matter in the present invention, herein a preferred embodiment is disclosed. It is noted that all objects contained in the embodiment are described in an appropriate ratio, scale, deformation or displacement quantities applicable to the scope of explanation purpose rather than the ratio of any actual elements of the present invention.

The object of the present invention is to provide an integrated health and entertainment management system built in a smart handheld device, wherein the handheld device, as availably made by the state-of-art technology, supports various wire and wireless communication means, including but not limited to infrared transmission, Bluetooth and WIFI in wireless form and USB connection technology in wired form. Since principles of signal transmission in foregoing communication means are well known and widely applied for a long time, there is no need to include further explanation herein. As for functions provided by said integrated health and entertainment management system contained in the smart handheld device are detailed in the following paragraphs by referring to accompanying drawings and preferred embodiments.

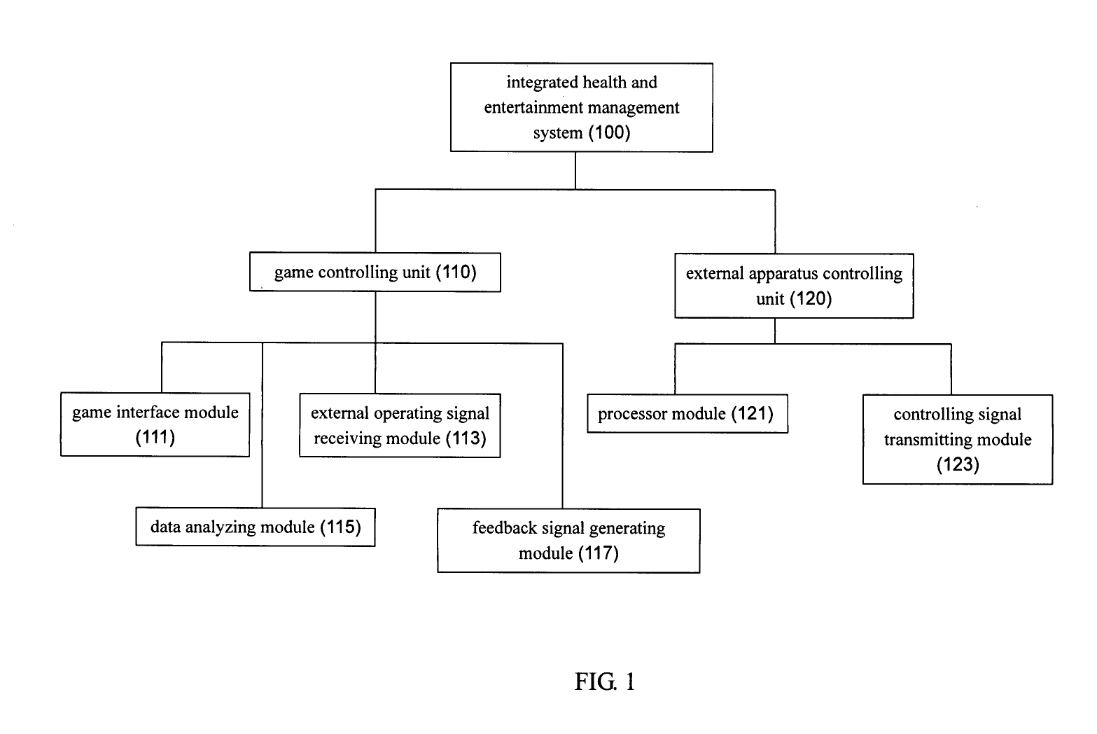

By referring to FIG. 1, a structural diagram of one embodiment of the integrated health and entertainment management system 100 built in the smart handheld device according to the present invention, said system 100 is consisting of two units; that is, a game controlling unit 110 and an external apparatus controlling unit 120. The layout and function of said elements in the said system are described as set forth in the following paragraphs by referring to FIG. 1.

The game controlling unit 110 contains a game interface module 111 for providing an interface in a display unit of the smart handheld device; an external operating signal receiving module 113 for receiving an operating signal 901 from an external exercise apparatus 900, the operating signal 901 being a resultant of real-time operation made by a user toward the external exercise apparatus 900; a data analyzing module 115 that compares the operating signal 901 with a default, then accordingly generates a comparison result 1151, and transforms the comparison result into a real-time dynamic image of a target in said interface; and a feedback signal generating module 117 that produces a feedback signal 1171 subject to the comparison result 1151.

Therein, the external exercise apparatus 900 can be a physical training equipments, including but not limited to a weight training apparatus, a motorized treadmill, a freewheeler, an upright cycle, an ellipse slider or a stepping exerciser. The so-called “real-time dynamic image of a target in said interface” means to visualize the operation made by the user toward the external exercise apparatus 900 in the game scenes, and thus allowing the target to complete missions or clear stage challenges preset in the interface. In addition to the visualization, the audio and video transmitting functions in the smart handheld device deliver amusing, indicating or encouraging sound effects, thereby enhancing the user's entertaining sensation during operating the external exercise apparatus 900.

For instance, when the external exercise apparatus 900 is a freewheeler, inputting the user's RPM, a resultant from user's cycling speed toward the freewheeler, into a racing game programmed in the smart handheld device, such input is interactively responded to as acceleration in gas pedal, speed changing and even an engine rolling sound. While a motorized treadmill operates as the external exercise apparatus 900, the user's operation toward the treadmill generates an operating signal that serves as a reference for determining if the user needs to adjust the resistance, speed and inclined angle of a running belt of the treadmill, or for changing to other training programs. Thereby increasing the user's joy and continuity in taking exercise through following an exercise journey designed in said system of the external exercise apparatus 900.

The external apparatus controlling unit 120 includes a processor module 121 generating a controlling signal 1211 subject to a predetermined training program and then adjusting the controlling signal 1211 according to the feedback signal 1171, so as to change the predetermined training program; and a controlling signal transmitting module 123 for sending out the controlling signal 1211 that drives the external exercise apparatus 900 to execute the preset or changed training programs.

For example, when the comparison result 1151 comes from the data analyzing module 115 showing that the user has smoothly completed missions, such as clearing a stage, in the game for an operating period, the feedback signal generating module 117 automatically adopts the feedback signal 1171 and rises the difficulty level in the preset training program to promote more challenges in the game, so that keeping the user's intension to take exercise continually. While the comparison result 1151 shows a contrary to aforesaid situation, the feedback signal generating module 117 automatically employs the feedback signal 1171 and reduces the difficulty level in the preset training program, and thus allowing the user to gain confidence, so that enhancing the user's willingness to keep taking exercise continually without quitting for being frustrated due to falling stages in the game.

During the operation of said system, the signal is mainly transmitted through existing wire or wireless communication forms; in addition to that, the present invention fully utilizes the sensation (or detection) function of the smart handheld device. In the present invention, one embodiment discloses a gravity-sensing device provided in the smart handheld device, a G-SENSOR. When the external exercise apparatus 900 has a flexible operating device, such as a universal joystick as described in the aforesaid prior art Taiwan Patent No. 22388, the user may mount the smart handheld device directly onto the joystick or physically connect the smart handheld device with the joystick, so that the G-SENSOR of the smart handheld device detects the motion of the joystick, then generates a sensing signal. Then, the data analyzing module 115 receives and processes the sensing signal produced from the G-SENSOR and the operating signal 901 come from the external exercise apparatus 900, then accordingly generates a comparison result 1151 that drives the target in the game. In other words, the data analyzing module 115 receives the sensing signal from the G-SENSOR of the smart handheld device and transforms therefrom a corresponding motion of the target. Again, in the racing game example, when the user's RPM serving as the operating signal 901 is input into the external exercise apparatus 900 and received by the data analyzing module 115, a motion change made by the user through the joystick directly activities the G-SENSOR of the smart handheld device and is transformed into a sensing signal. The sensing signal is processed along with the operating signal 901 by the data analyzing module 115, then accordingly generates a comparison result 1151, and changes the racing speed subject to the operating signal 901 and swifts the orientation according to the sensing signal. The present invention provides a more interactive and visual operation in some operating items in the integrated health and entertainment management system of the smart handheld device, thus increasing more fun in playing games.

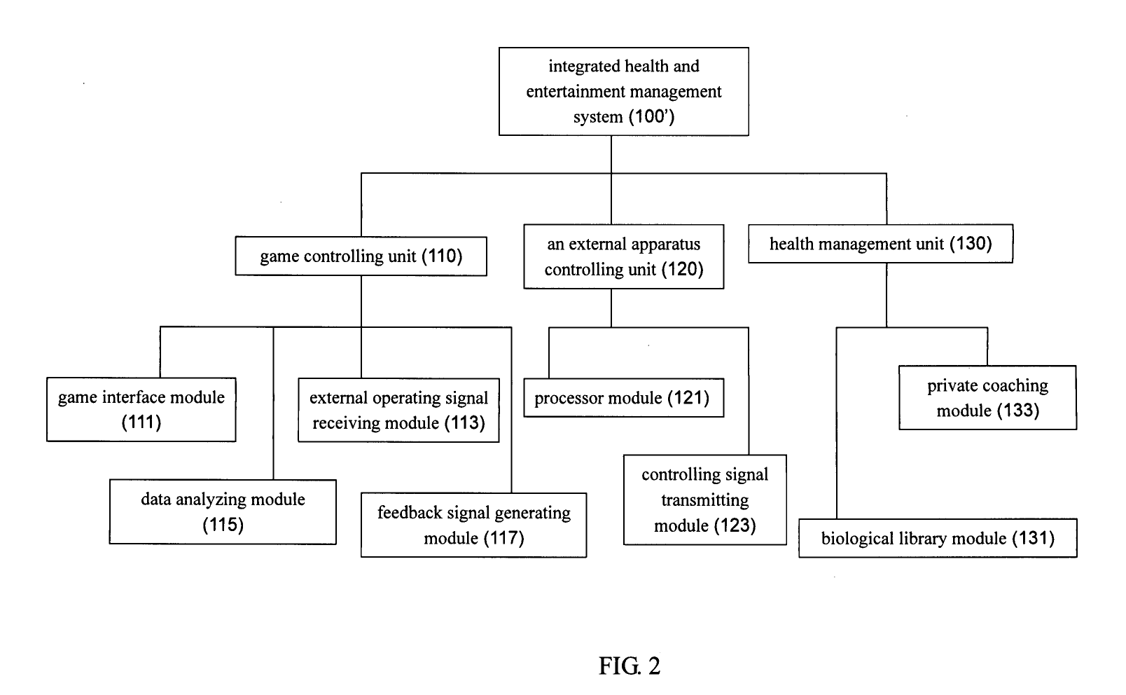

In another embodiment of the present invention, as shown in FIG. 2, the integrated health and entertainment management system 100′ built in the smart handheld device also contains a health management unit 130 consisting of a biological library module 131 and a private coaching module 133. Wherein, the biological library module 131 is loaded with the user's physiological information including but not limited to age, sex, height, weight and/or personal medical history, and can be updated by inputting any changes to above-mentioned information in the smart handheld device. The private coaching module 133 calculates a matching physical fitness standard and a training program on the basis of the user's physiological information, then suggests moderate sport items and intensity thereof, and evaluates the exercise outcome of user following such suggestion made by the private coaching module 133.

The health management unit 130 can connect with an external database 800 through any communication forms supported by the smart handheld device, such as Centers for Disease Control, R.O.C., and the private coaching module 133 can calculate a physical fitness standard and a training program subject to the data collected from the external database 800 and suggest moderate sport items and intensity thereof.

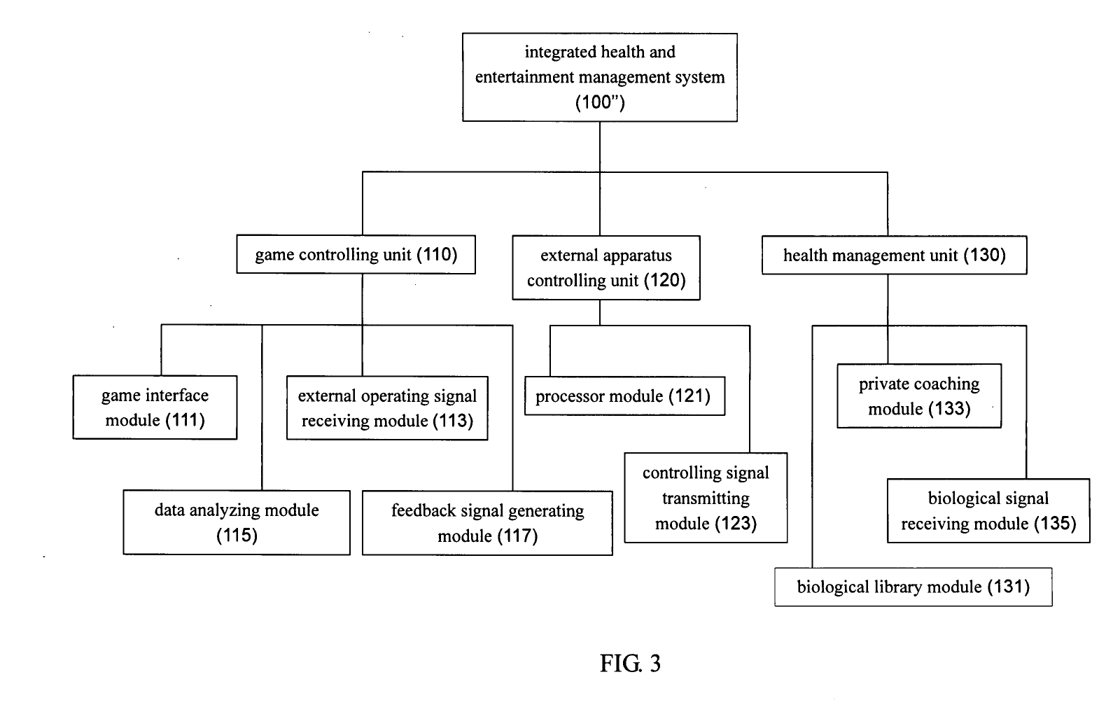

In another embodiment of the present invention, as shown in FIG. 3, the health management unit 130 in the integrated health and entertainment management system 100″ of the smart handheld device further comprises a biological signal receiving module 135 that is coupled with a biological signal monitoring device 700 for receiving a biological signal 701 from the biological signal monitoring device 700 and feedbacks the biological signal 701 to the health management unit 130 for being as a reference to the private coaching module 133 during evaluating exercise outcome of the user, so that the private coaching module 133 adjusts the sport items and intensity thereof in the exercise journey in accordance with the physical fitness standard and the training programs. That is, when the private coaching module 133 determines that the biological signal 701 is higher than an upper threshold, the private coaching module 133 triggers the processor module 121 to adjust the controlling signal 1211 for decreasing difficulty level in the predetermined training program while the biological signal 701 is lower than a lower threshold, the private coaching module 133 triggers the processor module 121 to adjust the controlling signal 1211 for increasing difficulty level in the predetermined training program. The aforesaid biological signal monitoring device 700 is including but not limited to a heartbeat counter, a tonometer and a respirometer sending the biological signal 701 in forms of heartbeat rates, blood pressure values and breathing rates.

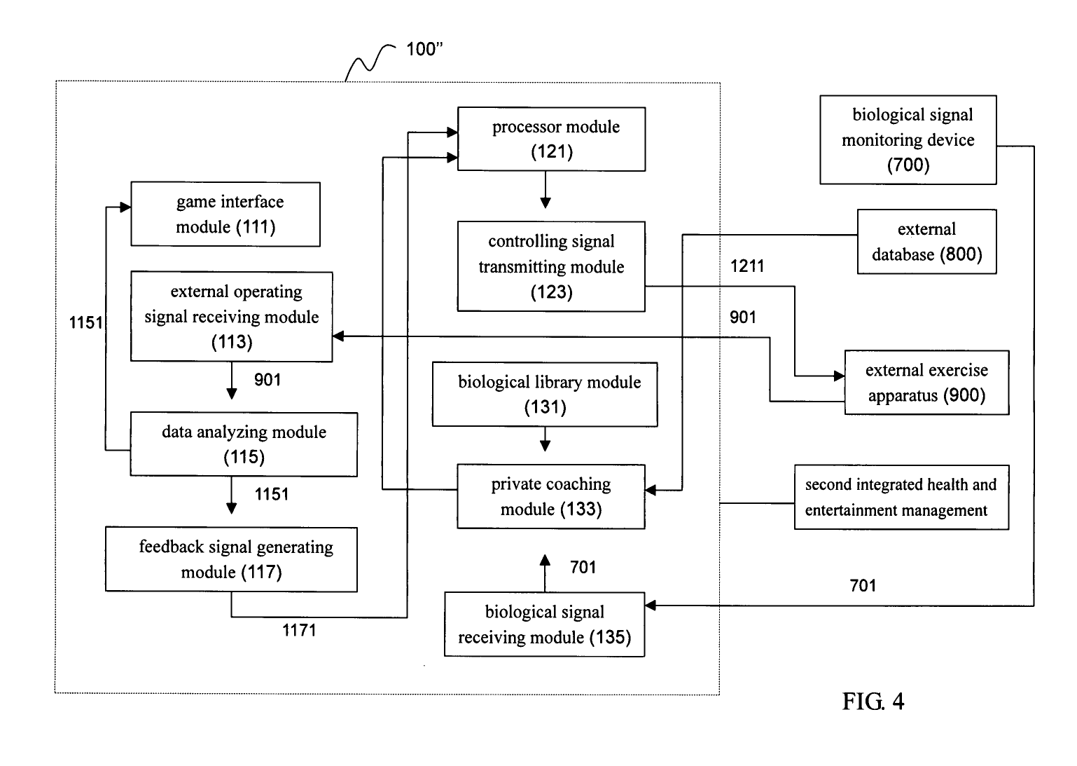

In details, the health management unit 130 calculates a maximum heart rate (MHR) according to the biological signal 701, provides an exercise suggestion and drives the processor module 121 in the external apparatus controlling unit 120 to change the controlling signal 1211 so as to adjust the predetermined training program. For example, when the external exercise apparatus 900 is a treadmill and the biological signal monitoring device 700 is a heart monitor, the biological signal receiving module 135 receives and analyzes the biological signal 701 that indicates a value approximate to MHR of the user, the health management unit 130 feedbacks the biological signal 701 to the processor module 121 in the external apparatus controlling unit 120 so as to make the controlling signal transmitting module 123 send out the adjusted controlling signal 1211 for reducing inclined angle and speed of the running belt and lowering the difficulty level of game and sport intensity for preventing the user from over fatigue. On the contrary, when the biological signal receiving module 135 receives and analyzes the biological signal 701 that indicates a value far below sufficient sport intensity, the health management unit 130 feedbacks the biological signal 701 to the external apparatus controlling unit 120 and drive the processor module 121 to adjust the controlling signal 1211 for increasing inclined angle and speed of the running belt and boosting the difficulty level of game and sport intensity, thereby forcing the user to work on and exercise effectively. The signal correlation between foregoing modules and unit is depicted in the FIG. 4.

Furthermore, the smart handheld device having said integrated health and entertainment management system may further combine with another smart handheld device having the same integrated health and entertainment management system thus allowing any of users of said smart handheld devices to operate a target that displays simultaneously in the interface in both smart handheld devices.

The health management unit 130 further comprises an identification module (not shown) for allowing more than one user to establish exclusive personal physiological information in the biological library module 131 and to update such information through the smart handheld device. Thus the system can support several users with one said portable smart handheld device.

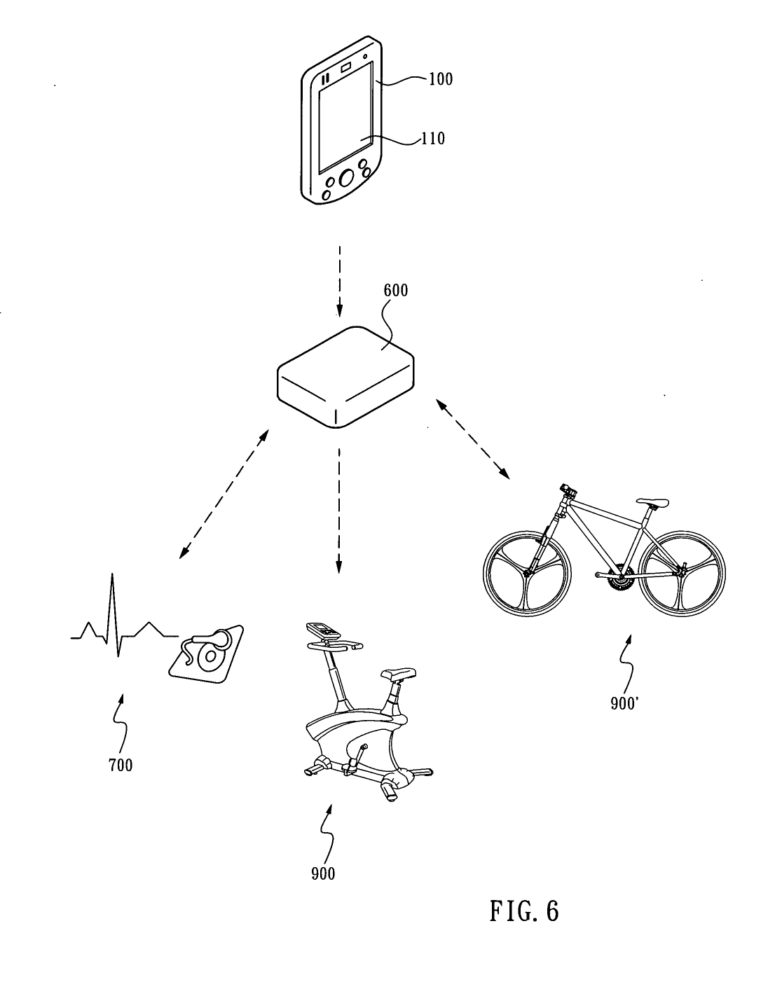

In above context, the major purpose of the present invention is to realize an application of the integrated health and entertainment management system in the existing smart handheld devices. In implementation of the present invention, some modules can be provided in the form of an auxiliary hardware subject to the requirements of the smart handheld device, and the difference between the personal handheld device 100 and 100′ is shown in the FIG. 1 through FIG. 2. While in FIG. 6, an auxiliary interface 600 can be a known communication interface or a sensing interface or some equivalents.

Therefore, the smart handheld device in the present invention is portable, equipped with a sensing device and excellent in communication, capable of connecting with various external exercise apparatuses and providing a highly efficient and interactive entertaining exercise pattern encouraging the users to take exercise continually and thus facilitating physical health and disease prevention.

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention rather than limit the scope of the present invention. Accordingly, the foregoing description and drawings are by way of example only.

What is claimed is:

1. An integrated health and entertainment management system for a smart handheld device that supports at least one communication means, the integrated health and entertainment management system comprising:
a game controlling unit, having:
a game interface module for providing an interface in a display unit of the smart handheld device;
an external operating signal receiving module for receiving an operating signal from an external exercise apparatus, the operating signal being a resultant of real-time operation made by a user toward the external exercise apparatus;
a data analyzing module for comparing the operating signal with a default, then accordingly generating a comparison result, and transforming the comparison result into a real-time dynamic image of a target in said interface;
a feedback signal generating module for producing a feedback signal according to the comparison result; and
an external apparatus controlling unit, having:
a processor module for generating a controlling signal according to a predetermined training program and adjusting the controlling signal according to the feedback signal, so as to change the predetermined training program; and
a controlling signal transmitting module for sending out the controlling signal to drive the external exercise apparatus to execute the unchanged or changed predetermined training program.

2. The integrated health and entertainment management system of claim 1, the integrated health and entertainment management system further includes a health management unit, having:
a biological library module to be loaded with the user's physiological information and updated through the smart handheld device; and
a private coaching module for calculating a matching physical fitness standard and a training program according to the user's physiological information.

3. The integrated health and entertainment management system of claim 2, wherein the health management unit further includes a biological signal receiving module that receives a biological signal from a biological signal monitoring device and feedbacks the biological signal to the health management unit for being as a reference to the private coaching module, when the private coaching module determines that the biological signal is higher than an upper threshold, the private coaching module triggering the processor module to adjust the controlling signal so as to decrease difficulty level in the predetermined training program, and when the biological signal is lower than a lower threshold, the private coaching module triggering the processor module to adjust the controlling signal so as to increase a difficulty level in the predetermined training program.

4. The integrated health and entertainment management system of claim 3, wherein the biological signal monitoring device includes a heartbeat counter, a tonometer and/or a respirometer sending the biological signal in forms of heartbeat rates, blood pressure values and breathing rates, and the upper and the lower thresholds are heartbeat rates, blood pressure values and breathing rates.

5. The integrated health and entertainment management system of claim 1, wherein the communication means of the smart handheld device includes infrared transmission, Bluetooth, WIFI or USB connection technology.

6. The integrated health and entertainment management system of claim 1, wherein the smart handheld device is equipped with a gravity-sensing device, and the external exercise apparatus has a flexible operating device, when the flexible operating device is physically connected with the smart handheld device, the user's operation toward the flexible operating apparatus being detected and directly effecting on the gravity-sensing device and the gravity-sensing device generating a sensing signal to be received and processed by the data analyzing module for generating the comparison result.

7. The integrated health and entertainment management system of claim 2, wherein the health management unit connects to an external database through the communication means supported by the smart handheld device, and the data collected from the external database is referred by the private coaching module in calculating a matching physical fitness standard and a training program and in suggesting moderate sport items and intensity thereof.

8. The integrated health and entertainment management system of claim 2, wherein the user's physiological information includes age, sex, height, weight and/or personal medical history.

9. The integrated health and entertainment management system of claim 1, wherein the external exercise apparatus is a physical training equipment, including a weight training apparatus, a motorized treadmill, a freewheeler, an upright cycle, an ellipse slider or a stepping exerciser.

10. The integrated health and entertainment management system of claim 1, wherein the smart handheld device having said integrated health and entertainment management system further combines with another smart handheld device having the same integrated health and entertainment management system, thus allowing any of users of said smart handheld devices to operate a target that displays simultaneously in the interface in both smart handheld devices.

11. The integrated health and entertainment management system of claim 2, wherein the health management unit further comprises an identification module for allowing more than one user to establish exclusive personal physiological information in the biological library module and to update such information through the smart handheld device.
